(19) United States
(12) Patent Application Publication
(10) Pub. No.: US 2008/0275651 A1
(43) Pub. Date: Nov. 6, 2008

(54) METHODS FOR INFERRING THE PRESENCE OF A PROTEIN IN A SAMPLE

(76) Inventors: Predrag Radivojac, Bloomington, IN (US); Randy J. Arnold, Bloomington, IN (US); Haixu Tang, Bloomington, IN (US); Pedro Alves, Hamden, CT (US); Yong Li, Bloomington, IN (US)

Correspondence Address: BARNES & THORNBURG LLP, 11 SOUTH MERIDIAN, INDIANAPOLIS, IN, 46204, US

(21) Appl. No.: 11/923,493

(22) Filed: Oct. 24, 2007

Related U.S. Application Data
(60) Provisional application No. 60/853,996, filed on Oct. 24, 2006.

(51) Int. Cl. G01N 33/48; G06F 17/18

(52) U.S. Cl. 702/19; 702/179

(57) ABSTRACT

A method of inferring presence of at least one protein in a sample includes entering a peptide training data set into a statistical inference model. The method further includes training the statistical inference model with the peptide training data set. The method further includes determining predicted detectability of at least one peptide present in the sample with the trained statistical inference model. The method further includes inferring the presence of the at least one protein in the sample based upon the determined predicted detectability. Methods for quantifying proteins present in a sample are also disclosed.

### CLAIM OF PRIORITY

This Application claims priority to U.S. Provisional Patent Application Ser. No. 60/853,996 filed on Oct. 24, 2006, the entirety of which is incorporated by reference herein.

### FIELD OF THE INVENTION

The invention generally relates to proteomics, and more specifically to protein analysis based upon peptide data.

### BACKGROUND

Rapid and reliable identification of thousands of peptides from a complex protein mixture sample using liquid chromatography tandem mass spectrometry (LC/MSMS) and other MS related technologies has established the foundation of high throughput proteomics experiments. Label-free protein quantification approaches attempt to quantify relative protein abundances directly from high-throughput proteomics analyses without applying labeling techniques. One approach to label-free protein quantification in high-throughput proteomics experiments is based solely on peptide identification, a method that has previously been shown to be quite reliable, by learning and applying peptide features to increase the reliability and accuracy of protein quantification.

### SUMMARY

According to one aspect of the disclosure, a method of inferring presence of at least one protein in a sample may include entering a peptide training data set into a statistical inference model. The method may further include training the statistical mode with the peptide training data set. The method may further include determining predicted detectability of at least one peptide present in the sample with the trained statistical inference model. The method may further include inferring the presence of the at least one protein in the sample based upon the determined predicted detectability.

According to another aspect of the disclosure, a method of quantifying proteins present in a sample may include entering a peptide training data set into a statistical inference model. The method may further include training the statistical inference model with the peptide training data set. The method may further include determining predicted detectability of at least one peptide present in the sample with the trained statistical inference model. The method may further include quantifying proteins present in the sample with an analyzer based upon the determined predicted detectabilities.

According to another aspect of the disclosure, a method of inferring presence of at least one protein in a sample may include analyzing the sample to produce a set of peptide data. The method may further include identifying peptides in the sample based upon the peptide data. The method may further include comparing the identified peptides to predetermined peptide detectabilities for all proteins containing at least one identified peptides. The method may further include inferring the presence of at least one protein in the sample based upon the comparison between the identified peptides and the predetermined peptide detectabilities.

According to another aspect of the disclosure, a method of quantifying at least one protein in a sample may include analyzing the sample to produce a set of peptide data. The method may further include identifying peptides in the sample based upon the peptide data. The method may further include comparing the identified peptides to predetermined peptide detectabilities for all proteins containing at least one identified peptides. The method may further include quantifying at least one protein in the sample based upon the comparison between the identified peptides and the predetermined peptide detectabilities.

### BRIEF DESCRIPTION OF THE FIGURES

FIG. 1(a) shows predicted detectabilities of tryptic peptides from each protein of a particular sample;

FIG. 1(b) shows the minimum acceptable detectability for identified peptides (MDIP) in various samples;

FIG. 2 shows the MDIP for hemoglobin samples;

FIG. 3(a)-(c) shows scatter plots of pairwise comparisons of MDIP scores between any two experiments;

FIG. 4 shows a detectability plot of a hypothetical proteins broken up into tryptic peptides;

FIG. 5(a)-(b) shows pairwise comparison of all proteins in an IPI rat database in which proteins share at least one identified peptide;

FIG. 6 shows a detectability plot of a hypothetical protein consisting of 8 tryptic peptides from two shotgun proteomics experiments;

FIG. 7(a) is a protein configuration graph;

FIG. 7(b) is a protein configuration graph illustrating a Bayesian model for protein inference;

FIG. 7(c) is a protein configuration graph illustrating another Bayesian model for protein inference;

FIG. 8 shows illustrative results of a method described herein;

FIG. 9 is a flowchart illustrating a method for inferring presence of at least one protein in a sample; and

FIG. 10 is another flowchart illustrating a method for inferring presence of at least one protein in a sample.

### DETAILED DESCRIPTION OF THE DISCLOSURE

### Peptide Detectability

There are four classes of factors that govern the likelihood of observing a peptide in a proteomics experiment: (i) the chemical properties of the peptide (and its parent protein); (ii) the limitation of the peptide identification protocol, including the pre-processing of the sample, the mass spectrometry (MS) instruments and software tools used for MS analysis; (iii) the abundance of the peptide in the sample; and (iv) the other peptides present in the sample that compete with this peptide in the identification procedure. The detectability of a peptide may be defined as the probability that the peptide will be observed in a standard sample analyzed by a standard proteomics routine.

In a number of illustrative experiments, data is investigated from samples treated by trypsin digestion followed by reversed-phase liquid chromatography tandem mass spectrometry (LC/MS) in an ion trap and searched against known protein sequences using Mascot. The term “standard sample” in this for purposes of this illustrative experiment means that the sample has a fixed number of different proteins (peptides) and they are mixed at the same fixed concentration (e.g., 1 pmol/injection). It should be noted that by this definition, peptide detectability is an intrinsic property of a peptide that is determined by its primary sequence as well as its location within the context of the entire protein. Peptides with higher detectabilities have a greater chance of being identified than those with lower detectabilities. As a result, if a peptide with low detectability is identified in a sample, it indicates that this peptide (or the protein this peptide is from) has a high abundance; if a peptide with high detectability is missed (not identified) in a sample, it indicates that this peptide (or the protein this peptide is from) has a low abundance. In addition, a situation in which a peptide with very low detectability is identified, while those with higher detectabilities are not, suggests a false positive identification. Therefore, the notion of peptide detectability may be used to establish a direct correlation between peptide identification and protein identification/quantification.

Given a protein, it is believed that the detectability of all tryptic peptides can be predicted from their sequences. It may be, however, important to generate a sample that satisfies the standard conditions as described above, as the learning set for such a prediction. In one illustrative experiment, an artificial sample (see sample B) mixed from 12 model proteins in the similar concentration (1 pmol/microliter) was prepared and analyzed using LC/MS and the identification results were used as a learning data set for a predictor of peptide detectability in LC/MS experiments.

Four groups of data sets of mass spectra were used in a number of illustrative experiments. The first group (data set A) was generated as a standard protein mixture consisting of 12 model proteins and 23 model peptides mixed at similar concentrations from 73 to 713 nM for proteins and from 50 to 1800 nM for peptides. The second group consisted of six data sets

TABLE 1

| Protein | SwissProt ID | MW (kDa) | B₁ | B₂ | B₃ | B₄ | B₅ | B |
|---|---|---|---|---|---|---|---|---|
| Serum albumin, bovine | P02769 | 66.4 | 3000 | 300 | 1000 | 30 | 100 | 1000 |
| Myoglobin, horse | P68082 | 17.0 | 3000 | 300 | 1000 | 30 | 100 | 1000 |
| Beta-casein, bovine | P02666 | 23.6 | 1000 | 3000 | 100 | 300 | 30 | 1000 |
| Catalase, bovine | P00432 | 59.8 | 1000 | 3000 | 100 | 300 | 30 | 1000 |
| Lactoferrin, bovine | P24627 | 76.1 | 300 | 30 | 3000 | 100 | 1000 | 1000 |
| Lysozyme, chicken | P00698 | 14.3 | 300 | 30 | 3000 | 100 | 1000 | 1000 |
| Alpha-casein, bovine | P02662 | 23.0 | 100 | 1000 | 30 | 3000 | 300 | 1000 |
| Pyruvate kinase, rabbit | P11974 | 57.9 | 100 | 1000 | 30 | 3000 | 300 | 1000 |
| Ovalbumin, chicken | P01012 | 42.8 | 30 | 100 | 300 | 1000 | 3000 | 1000 |
| DNase I, bovine | P00639 | 29.1 | 30 | 100 | 300 | 1000 | 3000 | 1000 |
| RNase A, bovine | P61823 | 13.7 | 30 | 100 | 300 | 1000 | 3000 | 1000 |
| Hemoglobin alpha, human | P69905 | 15.1 | 2000 | 2000 | 2000 | 2000 | 2000 | 2000 |
| Hemoglobin beta, human | P68871 | 15.9 | 2000 | 2000 | 2000 | 2000 | 2000 | 2000 |

(data sets B and B₁-B₅), prepared in a laboratory, each representing a mixture of the same 13 model protein chains. To mimic a similar peptide competition environment in the LC/MS analysis, similar total amounts of protein were intentionally mixed in each sample as indicated in Table 1. The third group is a data set (data set C) generated from a real rat proteome, as described later. The last group consists of three data sets (data sets D₁-D₃) representing three replicate analyses of the fruit fly head proteome. With the exception of data set C, all samples were reduced and alkylated with iodoacetamide prior to trypsin digestion. The rat samples were digested in the presence of an acid-labile surfactant. MS experiments in the illustrative experiment were carried out on an ion trap mass spectrometers, such as a 3-Dion trap (data sets A, C, and D) and a linear ion trap (data set B). The low m/z cut-off was between 250 and 400, and the high m/z cut-off was between 1500 and 2000 for all experiments.

Due to the large differences in protein concentrations in the whole cell lysates, included in this illustrative experiment and learning procedures are only those proteins whose coverage of identified peptides was 10% or higher. In the case of the synthetic sample, one of the proteins contained only one identified peptide and was also removed from the subsequent analysis. The total number of protein chains, the number of tryptic peptides and the number of identified peptides in each data set are summarized in Table 2.

TABLE 2

| Data set | Protein chains | Total tryptic peptides | Identified peptides |
|---|---|---|---|
| A | 11 | 346 | 100 |
| B | 13 | 294 | 91 |
| C | 124 | 3403 | 359 |
| D₁-D₃ | 200 | 3722 | 526 |

Given an unseen n-residue long protein sequence S=s₁s₂ . . . sₙ and a database of peptides already detected by Mascot with high confidence, a model may be constructed that may approximate the probability of detecting any particular tryptic peptide from S with the same confidence. This probability may be denoted as P(score(s_{[i,j]})≧t|S), where s_{[i,j]}=sᵢsᵢ₊₁ . . . sⱼ represents a residue sequence of a tryptic peptide from S and t is defined as an appropriately selected Mascot threshold (by default 40 in the exemplary experiments). In the case when a Pro residue directly follows a basic residue (Arg or Lys), the peptide was extended until the first accessible Arg/Lys or until the C-terminus. As previously mentioned, in order to reduce the dependency of the detectability on the concentration of the protein in a cell, only proteins with ≧10% sequence coverage of the detected peptides were used in the illustrative experiments, however it should be appreciated that this threshold may be greater or lesser than that used. In the illustrative experiments, all peptides whose m/z was outside of the instrument range were eliminated from training and testing as trivial.

To enable learning in the illustrative experiments, each input peptide sequence s_{[i,j]} was represented by a fixed-length vector of real- or discrete-valued features. Two groups of features were considered: those that depend on s_{[i,j]} only and those that also depend on the flanking regions. Thus, an identical peptide observed in the contexts of different sequence neighborhoods will in general have different detectability. The following groups of features were constructed from s_{[i,j]}: (i) amino acid compositions in the peptide; (ii) length of the peptide, i.e. j−i+1; (iii) ion mass m(s_{[i,j]}); (iv) N- and C-terminal residues, sᵢ and sⱼ; (v) sequence complexity; (vi) physicochemical properties averaged over the entire peptide—aromatic content and hydrophobicity; and (vii) predictions obtained from various bioinformatics tools and averaged over s_{[i,j]}—namely, protein flexibility predictors, hydrophobic moment, and predictions of intrinsic disorder. Since the detectability of the peptide may also be influenced by the neighboring regions, the composite features from (vii) were averaged over the regions of ±5, +10, and ±15 residues flanking both sides of s_{[i,j]}. In addition, the residue at position sⱼ₊₁ was also accounted for. Individual amino acids were encoded using orthogonal data representation while the compositional features were encoded by real values. Overall, the total number of features was 175 for the illustrative experiments. A binary class label was added to each feature vector; 1 (positive) for a detected peptide and 0 (negative) otherwise.

To build predictors of peptide detectability for this illustrative experiment, ensembles of 30, two-layer feed-forward neural networks trained using a resilient backpropagation algorithm were employed. However, it should be appreciated the number of models in an ensemble may be more or less than those used in the illustrative experiment. It should also be appreciated that various other statistical inference models may be used such as machine learning models, for example. These models include, but are not limited to, support vector machines, logistic regression, Bayesian networks, etc and also incorporate methodology for normalization and dimensionality reduction. Due to the asymmetric class sizes and small positive set (such as detected fragments), each in one illustrative experiment the network was trained on a balanced selection of positive and negative examples, however variations thereof may be implemented, as well. Each individual training set contained all the examples from the positive class and the same number of randomly selected negative examples. The network contained 1 output neuron, while the number of hidden neurons h was varied from hε{1, 2, 4}. All neurons contained the logistic activation function. Prior to the network training, unpromising features were eliminated using the t-test filter in which features whose p-values were above a given threshold t_{f}_{ₛ} were eliminated. The threshold t_{f}_{ₛ} for feature selection was varied from t_{f}_{ₛ}ε{0.01, 0.1, 1}. Note that in the case of t_{f}_{ₛ}=1, all features were retained. Finally, correlated features were removed by employing principal component analysis and retaining 95% of the variance. A validation set containing 20% of the training data was used for model selection and overfitting prevention for each of the training sets in the ensemble. Thus, the final prediction was averaged over 30 different models and a single estimated accuracy generated.

The performance of the predictor was evaluated within each data set (A to D) and also across various data sets. These two types of performance evaluation may be referred to herein as cross-validation and out-of-sample estimation, respectively. In the first case, a per protein 10-fold cross-validation was used. The entire set of available proteins D was first split into 10 non-overlapping sets {Dᵢ|i=1 . . . 10}. In each step i, dataset D−Dᵢ was used for training, while the prediction accuracy was estimated on the test set Dᵢ. The final performance estimates were obtained as averages over all 10 iterations. In the out-of-sample case, training and evaluating predictor performance on two independent experiments was of interest. In particular, a predictor was trained and optimized on one data set (for example, data set A) and then applied and evaluated on all other data sets (for example, data sets B, C and D). All twelve combinations were explored in the illustrative experiment.

In the illustrative experiments, sensitivity (sn) is defined as the fraction of detected peptides correctly predicted, and specificity (sp) is defined as the fraction of undetected peptides correctly predicted. Both were measured. Given sn and sp, the class-balanced accuracy may be calculated as accuracy=(sn+sp)/2. In this setup, a predictor always outputting the same class and a predictor outputting uniformly at random would have a balanced-sample accuracy of 50%. In addition to accuracy, the area under the ROC curve (AUC) using the trapezoid rule was estimated. Both accuracy and area under the curve appear to be essentially unaffected by the asymmetry in class sizes.

Analyzing features that discriminate between identified and unidentified peptides were also analyzed to provide insights into sequence and physicochemical properties governing peptide detectability. These features were selected using the standard two sample t-test on each feature independently. In particular, a feature was split into two 1-D samples according to the class label and a hypothesis that these samples were generated according to the same probability

TABLE 3

| Feature | Window | p-value | Correlation |
|---|---|---|---|
| Vihinen et al. flexibility | ±15 | 3.1 · 10⁻¹⁰ | − |
| Hydrophobic moment | ±15 | 6.0 · 10⁻¹⁰ | − |
| B-factor prediction | ±15 | 2.9 · 10⁻¹⁰ | − |
| VL2 disorder | ±15 | 1.3 · 10⁻⁷ | − |
| Sequence complexity | 0 | 1.8 · 10⁻⁷ | + |
| VL2V disorder | ±15 | 3.5 · 10⁻⁶ | − |
| VLXT disorder | ±15 | 4.1 · 10⁻⁶ | − |
| VL2S disorder | ±15 | 4.3 · 10⁻⁵ | − |
| VL3 disorder | ±15 | 5.5 · 10⁻⁵ | − |
| Composition of Lys | 0 | 3.3 · 10⁻⁴ | − |
| Mass/length ratio | 0 | 1.0 · 10⁻³ | − |
| VL2C disorder | ±15 | 4.1 · 10⁻³ | − |
| Composition of Val | 0 | 1.6 · 10⁻² | + |
| Length | 0 | 1.8 · 10⁻² | + |
| Composition of Gly | 0 | 2.1 · 10⁻² | + |

distribution was tested. Even though the features may not come from a Gaussian distribution, the t-test is known to be robust to violations of this assumption. In Table 3, a ranking is presented according to the increasing p-value of 15 individual features obtained on data set B that provided superior discrimination in this analysis.

Nine of these features contained in Table 3 were based on the overall properties of the peptide including its neighborhood, while the top ranked features based solely on the peptide itself were sequence complexity, its length, the mass/length ratio and presence of Lys, Val, and Gly. Other data sets had similar ordering of the features (data not shown). As a general rule, it is believed that peptides within flexible neighborhoods have lower detectability. On the other hand, presence of hydrophobic amino acids (Val, Gly) and peptide length were positively correlated with peptide detectability.

In this illustrative experiment, evaluation of the predictor was performed in two steps. In a first step, a 10-fold cross-validation was used to estimate the prediction accuracy on each data set. In a second step, performance evaluation was performed across data sets, as described above. The summary of systematic evaluations is shown in Table 4.

TABLE 4

| | | Training set | | | |
|---|---|---|---|---|---|
| accuracy/AUC | | A | B | C | D₁-D₃ |
| Test | A | 75.8/79.7 | 74.8/80.3 | 68.0/72.0 | 63.0/79.2 |
| set | B | 68.3/77.5 | 65.5/70.0 | 62.8/69.6 | 62.7/68.7 |
| | C | 66.7/74.6 | 66.8/73.5 | 75.0/84.0 | 68.0/78.1 |
| | D₁-D₃ | 78.7/86.5 | 73.1/79.0 | 79.9/87.6 | 86.8/93.0 |

Generally, results shown in Table 4 indicate that peptide detectability is influenced by its sequence and flanking regions from the parent protein. The data sets can be grouped into synthetic and whole cell, based on their out-of-sample performance. For example, best out-of-sample accuracy on data sets A and B was achieved when the training sets were B and A, respectively. Training on these synthetic data sets also achieved adequate performance even on data sets C and D, despite small training sizes. The best out-of-sample performance on data set C was achieved by training on data set D, while the best out-of-sample performance on data set D was achieved by training on C.

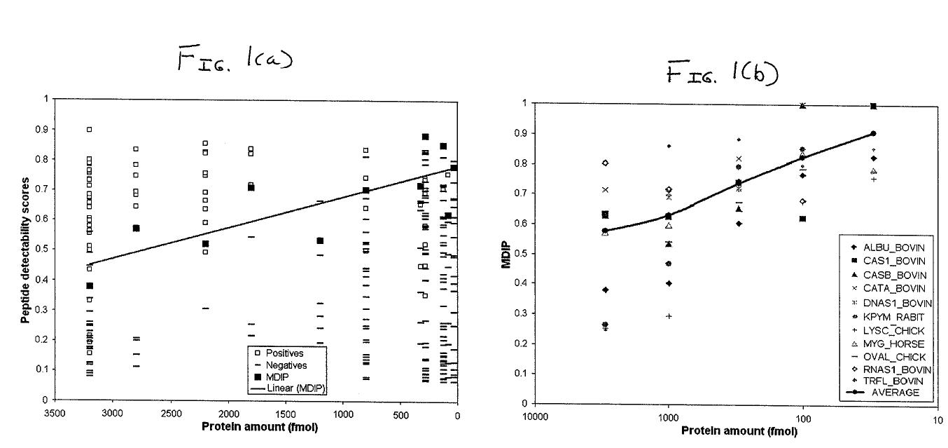

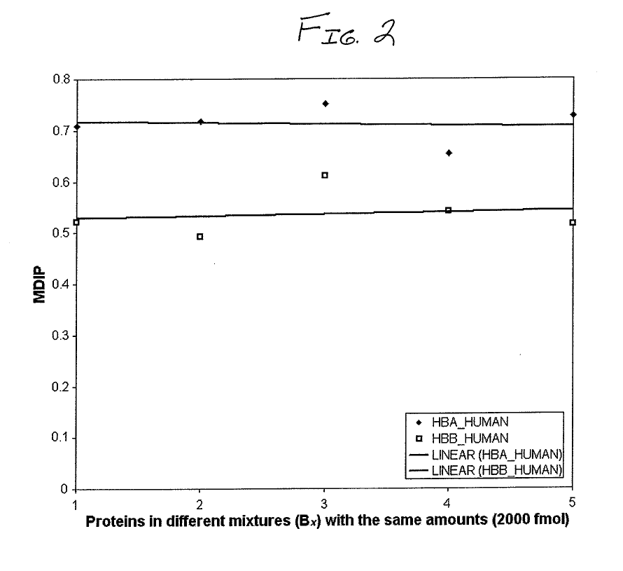

In the illustrative experiment, samples B₁-B₅ were analyzed using a predictor trained on sample B in which all chains were similarly abundant. FIG. 1(a) shows the predicted detectabilities of all tryptic peptides from each protein from sample B₁. Peptides from the same protein are shown in the same column, sorted by their detectabilities. Proteins were sorted by their relative abundances (concentrations) in the mixture. In FIG. 1(a), the identified peptides are shown as empty squares, while the missed peptides are shown as dashes for illustrative purpose. It is clear that, for each protein in sample B₁, the identified peptides tend to have higher detectabilities than those not identified. This is consistent to the prediction accuracy results as previously discussed. For each protein, its minimum acceptable detectability of identified peptides (MDIP), a cutoff value of detectability, which maximizes the sum of true positive and true negative rates, may be determined. For example, if all peptides from a protein are detected, the MDIP of this protein is set to 0, and if none of the peptides from a protein is detected, the MDIP of this protein is set to 1. It may be observed from FIG. 1(a) that the MDIP values, shown as solid squares, increase as the protein abundance decreases. This trend is approximated by a solid regression line (Linear (MDIP)). Similar results were obtained in the remaining samples B₂-B₅ (data not shown).

The MDIP was found for each protein in five different synthetic mixtures (B₁-B₅) and are shown in FIG. 1(b). Each column in FIG. 1(b) corresponds to a particular concentration and represents proteins from different experiments. For example, in the second column from the MDIP axis, the shaded diamond and circle represent proteins ALBU_BOVIN and KPYM_RABIT, respectively, both with concentration 1000 fmol. However, ALBU_BOVIN was mixed at this concentration in sample B₃, while KPYM_RABIT was mixed at concentration 1000 fmol in sample B₂ (see Table 1). Similar to the trend observed in FIG. 1(a), in FIG. 1(b) a linear relationship is observed between MDIP and protein concentration. Moreover, their relationships are generally similar from one protein to the next.

FIG. 2 shows the MDIP for hemoglobin A and hemoglobin B, which were mixed in the same amount in all experiments (see Table 1), across different samples. It shows low variation of MDIP, suggesting it is a robust measure of protein abundance.

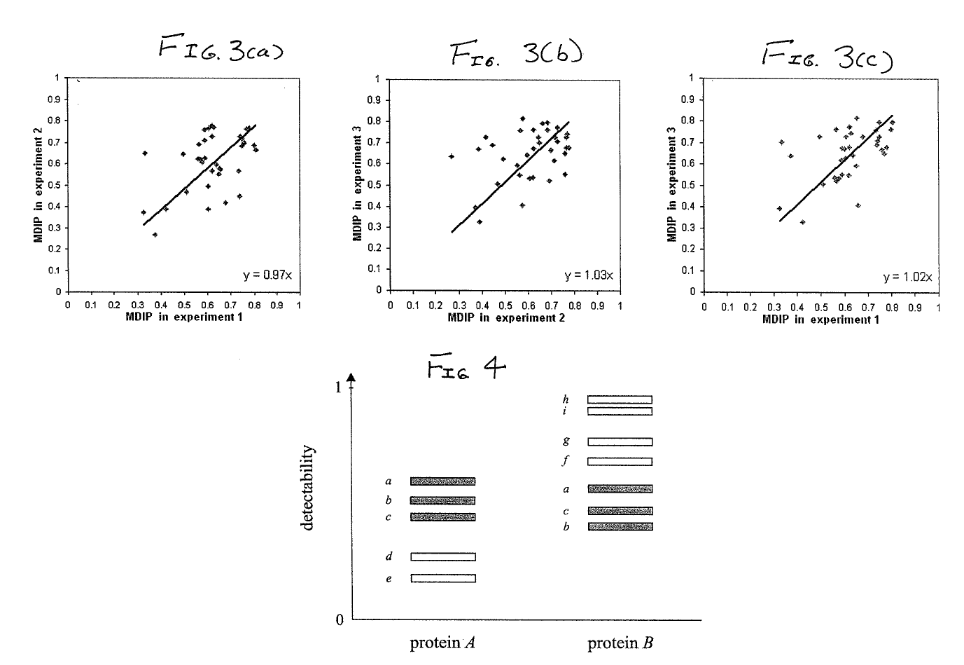

It may also be shown that MDIP may be used as a measure of protein quantification in high throughput proteomics experiments. Here, three replicate data sets (D₁-D₃) are used to demonstrate the robustness of the protein quantification method. Using the same predictor trained on data set B, the detectability is predicted of all proteins in D. melanogaster proteome. In each of the three experiments (D₁-D₃), computed the MDIP score was computed for each protein. FIG. 3 shows the scatter plots of pairwise comparisons of MDIP scores between any two experiments.

### Mass Spectrum Acquisition and Analysis

Mixtures of twelve standard proteins (listed in Table 1) were paired or triply-grouped such that the combined molecular weights in each group totaled about 80 to 90 kDa. Samples of each protein were prepared as stock solutions of 60, 20, and 2 micromolar concentration, or 90, 30, and 3 micromolar for the triply-grouped samples. Proteins were then mixed in various ratios such that the same molecular weight equivalent was present at 3000, 1000, 300, 100, and 30 fmol per microliter of final digestion solution, combined with buffer, reduced with dithiothreitol (DTT), alkylated with iodoacetamide (IAM), and digested at 37° C. for 18 hours. After acidification, samples were loaded onto a 15 mm by 100 micron i.d. trapping column packed with 5-micron BioBasic 18 particles with 300 angstrom pores. Peptides were separated using a 30-minute reversed-phase liquid chromatography gradient from 3% to 40% acetonitrile at 250 mL/min on a 12 to 15 cm, 75 micron i.d. capillary column pulled to a small (˜10 micron) tip and packed in-house with 5 micron C-18 coated particles. As peptides eluted from the column, they were electrosprayed into the source of a linear ion trap mass spectrometer, such as a Thermo Electron (San Jose, Calif.) LTQ, and analyzed by mass spectrometry and tandem mass spectrometry. By using dynamic exclusion, the mass spectrometer was limited to acquiring only one tandem mass spectrum for a given parent m/z over a 30-second window.

Rat brain regions (amygdala, caudate putamen, frontal cortex, hippocampus, hypothalamus, and nucleus accumbens) were digested separately with proteomics grade (modified) trypsin in the presence of an acid-labile surfactant. Tryptic peptides were separated by nano-flow reversed-phase liquid chromatography and electrosprayed directly into an ion-trap mass spectrometer, such as a ThermoFinnigan (San Jose, Calif.) LCQ Deca XP, for example, which recorded mass spectra and data-dependent tandem mass spectra of the peptide ions. Dynamic exclusion was employed to limit acquisition of tandem mass spectra for the same parent m/z over a 60-second window.

Drosophila genotype: elav-GAL4 (Stock number: Bloomington/458) flies were harvested and separated according to sex at day 1 of adult life. Flies were cultured on standard cornmeal medium and maintained at 25° C. Flies (n=250) were anesthetized with CO₂, flash frozen and decapitated with shaking in liquid N₂. Heads were collected on dry ice and stored at −80° C. Proteins were extracted using a mortar and pestle in 0.2 M phosphate buffer saline plus 8 M urea plus 0.1 mM phenylmethylsulfonyl fluoride (pH 7.0) solution. Proteins were centrifuged (15700 g at 4° C.) for 10 minutes and the supernatant was kept for the determination of protein concentration using Bradford assay. Extracted proteins were reduced with DTT, alkylated with IAM, and digested with TPCK-treated trypsin after diluting the urea to 2 M with 0.2 M Tris buffer (pH 8.0). Tryptic peptides were isolated by C-18 solid-phase extraction, vacuumed to dryness, and stored at −80° C. until future use. Peptides from each SCX fraction were separated by nano-flow reversed-phase liquid chromatography (15 cm×75 μm i.d. fused silica capillary column pulled to a fine tip and packed with 5 μm, 100 Å amino-terminated C18 packing material, eluted with a gradient from 5 to 45% acetonitrile at 250 mL/min). Eluting peptides were electrosprayed directly into the source of a Thermo Finnigan LCQ Deca XP ion trap mass spectrometer and analyzed by MS (m/z 250-1500) and data-dependent MS/MS on the three most intense ions.

In this illustrative experiment, tandem mass spectra were searched against protein sequences for the twelve known proteins (data set B), R. norvegicus in the Swiss-Prot database (data set C) or D. melanogaster (data set D) using a licensed copy of Mascot for peptide identification. Searches were performed with fixed modification of carbamidomethyl cysteine (where appropriate) and variable modifications of protein N-terminal acetylation and methionine oxidation selected and a maximum of one missed cleavage site. Mascot result files were parsed using a Protein Results Parser program to create training sets including all peptides with Mascot scores of 40 or higher for doubly-charged precursors. Peptides with Mascot scores below 40 were treated as negatives in the training sets.

### Shotgun Proteomics

Shotgun proteomics refers to the use of bottom-up proteomics techniques in which the protein content in a biological sample mixture is digested prior to separation and mass spectrometry analysis. Typically, liquid chromatography (LC) is coupled with tandem mass spectrometry (MS/MS) resulting in high-throughput peptide analysis. The MS/MS spectra are searched against a protein database to identify peptides in the sample. Currently, computer programs known in the art, such as Sequest and Mascot, are typically used for conducting peptide identification, both comparing experimental MS/MS spectra with in silico spectra generated from the peptide sequences in a database. In comparison to top-down proteomics techniques, shotgun proteomics may avoid the modest separation efficiency and poor mass spectral sensitivity typically associated with intact protein analysis, but it also encounters a new problem in data analysis, that of determining the set of proteins present in the sample based on the peptide identification results. While appearing trivial at the outset, it may be concluded that a protein is present in the sample, if and only if at least one of its peptides is identified. This conclusion is true, however, only when each identified peptide is unique, i.e. when it belongs to only one protein.

If some peptides are degenerate, i.e. shared by two or more proteins in the database, determining which of these proteins exist in the sample has multiple possible solutions. Indeed, tryptic peptides are frequently degenerate, especially for the proteome samples of vertebrates, which, due to recent gene duplications, often have a large number of paralogs. In addition, alternative splicing in higher eukaryotes results in many identical protein subsequences.

The following example illustrates the extent of peptide degeneracy in a real proteomics experiment. Of the 693 identified peptides from a real rat sample used in illustrative experiments described herein, 296 were unique and 397 were degenerate, when searched against the full proteome of R. norvegicus. These peptides can be assigned to a total of 805 proteins, of which only 149 proteins could be assigned based on the 296 unique peptides.

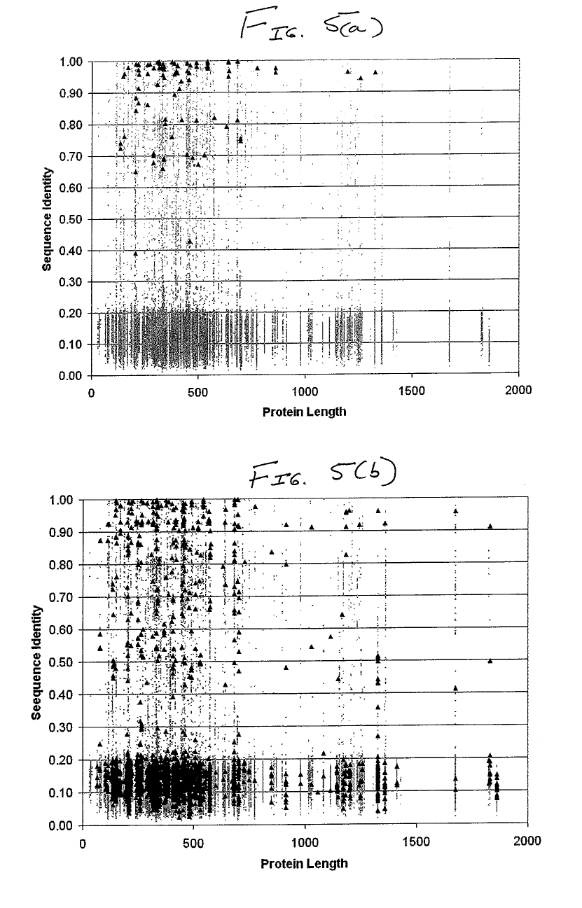

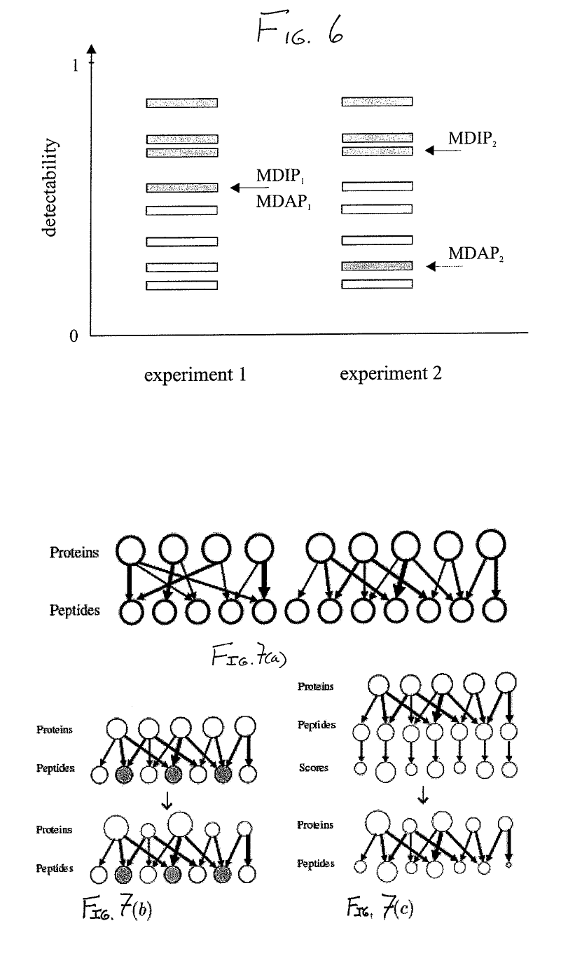

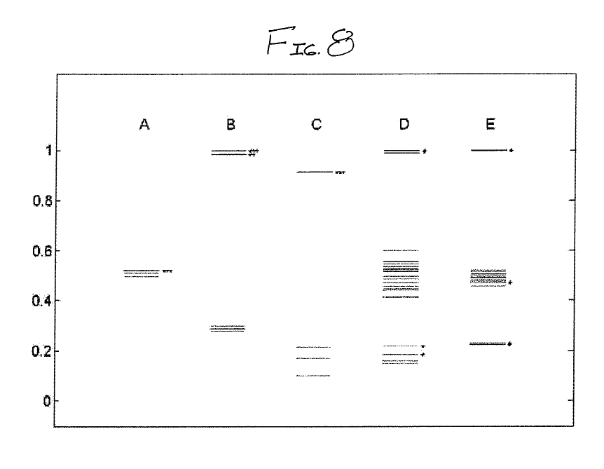

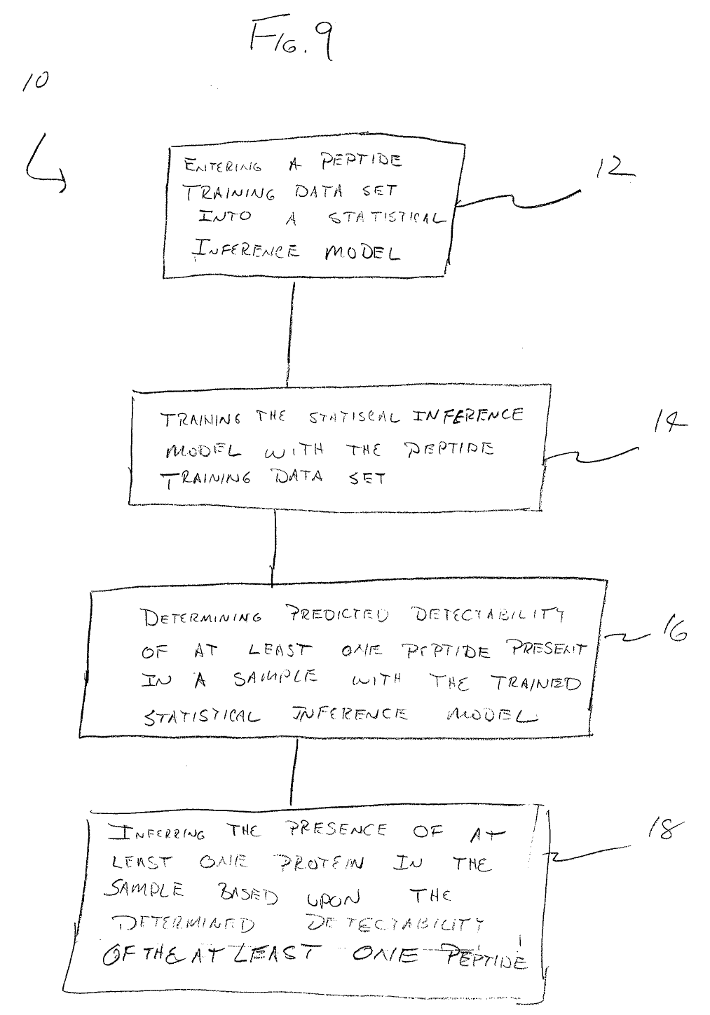

It should be appreciated by those of ordinary skill in the art the challenge of shotgun proteomics analysis, which has been formalized as the “protein inference problem.” In one illustrative experiment, the protein inference problem based on the concept of peptide detectability may be utilized. As previously described herein, the detectability of a peptide is defined as the probability of observing it in a standard proteomics experiment. It is believed that detectability is an intrinsic property of a peptide, completely determined by its sequence and its parent protein. The previously described illustrative experiment indicated that the peptide detectability may be estimated from the primary structure of its parent protein using a statistical inference model approach. The introduction of peptide detectability provides a new approach to protein inference, in which not only identified peptides but also those that are missed (not identified) are important for the overall outcome. FIG. 4 illustrates a utility of this particular idea. Assume A and B are two proteins sharing 3 degenerate tryptic peptides (a, b, and c, shaded bars). Each protein in FIG. 4 also has unique tryptic peptides (d, e, and f, g, h, i respectively, white bars). According to the original formulation of the protein inference problem, the identities of A and B cannot be determined since the only identified peptides are degenerate. However, if all the tryptic peptides are ranked in each protein according to their detectabilities (FIG. 4), it may be inferred that protein A is more likely to be present in the sample than protein B. This is because if B is present it is likely that peptides f-i would have been observed along with peptides a-c, which all have lower detectabilities than either f, g, h, or i. On the other hand, if protein A is present, peptides d and e may still be missed, which have lower detectabilities than peptides a-c, especially if A is at relatively low abundance. Thus, peptide detectability and its correlation with protein abundance provide a manner of inferring the likelihood of identifying a peptide relative to all other peptides in the same parent protein. This idea may then be used to distinguish between proteins that share tryptic peptides based on a probabilistic framework.

For purposes of illustration, consider an exemplary set of proteins P={P₁, P₂, . . . , P_{N}} such that each protein Pⱼ consists of a set of tryptic peptides {pⱼⁱ}, i=1, 2, . . . , nⱼ, where nⱼ is the number of peptides in {pⱼⁱ}. Suppose that F={f₁, f₂, . . . , f_{M}} is the set of peptides identified by some database search tool and that F⊂∪{pⱼⁱ}. Finally, assume each peptide pⱼⁱ has a computed detectability D(pⱼⁱ), for j=1, 2, . . . , N, and i=1, 2, . . . , nⱼ. D denotes the set of all detectabilities D(pⱼⁱ), for each i and j.

The goal of a protein inference algorithm is to assign every peptide from F to a subset of proteins from P which are actually present in the sample. This assignment may be termed as the correct peptide assignment. However, because in an actual proteomics experiment the identity of the proteins in the sample is unknown, it is difficult to formulate the fitness function that equates optimal and correct solutions. Thus, the protein inference problem can be redefined to find an algorithm and a fitness function, which results in the peptide-to-protein assignments that are most probable, given that the detectability for each peptide is accurately computed. In a practical setting, the algorithm's optimality can be further traded for its robustness and tractability.

If all peptides in F are required to be assigned to at least one protein, the choice of the likelihood function does not affect the assignment of unique (non-degenerate) peptides in ∪{pⱼⁱ}. On the other hand, the tie resolution for degenerate peptides will depend on all the other peptides that can be assigned to their parent proteins, and their detectabilities. In order to formalize this approach the following definitions may be used:

Definition 1: Suppose that the peptide-to-protein assignment is known. A peptide pⱼⁱε{pⱼⁱ} is considered assigned to Pⱼ if and only if pⱼⁱεF and D(pⱼⁱ)≧Mⱼ. Then, MⱼεD is called the Minimum Detectability of Assigned Peptides (MDAP) of protein Pⱼ.

Definition 2: A set of MDAPs {Mⱼ}_{j=1, 2, . . . , N} is acceptable if for each fεF, there exists Pⱼ, such that D(f)≧Mⱼ. Thus, any acceptable MDAP set will result in an assignment of identified peptides that guarantees that every identified peptide is assigned to at least one protein.

Definition 3: A peptide pⱼⁱ is missed if pⱼⁱ∈F and D(pⱼⁱ)≧Mⱼ.

Note that, due to the connection between peptide detectability and protein amount in the sample, peptides whose detectabilities are below Mⱼ are not considered missed. Thus, the protein inference problem may be formulated as follows:

Minimum missed peptide problem: Given N proteins, each consisting of nⱼ tryptic peptides, and a set of identified peptides F, find an acceptable set of MDAPs, {Mⱼ}_{j=1, 2, . . . , N}, which result in a minimum number of missed peptides.

If a protein does not exist in the sample, the MDAP Mⱼ needs to be assigned a value greater than the maximum detectability observed in Pⱼ. If a protein j is not present in the sample, Mⱼ is set to a maximum MDAP(=∞). Hence, only proteins whose Mⱼ≦1 are considered identified. Note that in nearly all practical cases the maximum MDAP can be set to 1, except when there is a peptide in ∪{pⱼⁱ} whose D(pⱼⁱ)=1. The relationship between the minimum missed peptide problem and the original minimum protein set problem is evidenced in the following theorem.

Theorem 1: Minimum missed peptide problem is NP-hard.

Proof: The minimum missed peptide problem can be reduced to the set-covering problem by setting D(pⱼⁱ)=0 for each i, j and adding a non-existing peptide with detectability of 1 to each protein. Minimizing the number of missed peptides now minimizes the number of covering subsets (proteins) in the solution set.

The data used in the illustrative experiments were that of data sets A, B, and D, as shown in Table 1. Data set B was from a mixture of twelve standard proteins was prepared at 1 μmol of final digestion solution for each protein except human hemoglobin which is at 2 μmol, combined with buffer, reduced, alkylated, and digested overnight with trypsin. Peptides were separated by nano-flow reversed-phase liquid chromatography gradient and analyzed by mass spectrometry and tandem mass spectrometry in a linear ion trap mass spectrometer, such as a Thermo Electron (San Jose, Calif.) LTQ, for example.

Data set D was generated using a complex proteome sample from R. norvegicus. Rat brain hippocampus samples were homogenized and separated by sedimentation in a centrifuge to produce four fractions enriched in nuclei, mitochondria, microsomes (remaining organelles), and the cytosol. Each subcellular fraction was subjected to proteolytic digestion with trypsin and analyzed by reversed-phase capillary LC tandem mass spectrometry using a 3-D ion trap, such as a ThermoFinnigan LCQ Deca XP, for example. Searches versus either the Swiss-Prot or the IPI rat database were performed for fully tryptic peptides using Mascot with a minimum score of 40 and allowing for N-terminal protein acetylation and methionine oxidation.

As previously above, the probability that a peptide will be identified in a standardized proteomics experiment is referred to as the peptide detectability. Using statistical inference model approaches the previously described illustrative experiment provided evidence that peptide detectability can be predicted solely from the amino acid sequence of its parent protein. A set of 175 features was constructed describing the peptide sequence itself as well as the regions upstream or downstream from the peptide. An ensemble of neural networks was then trained and evaluated. It was estimated that its balanced-sample accuracy at about 70% across training and test sets obtained from several independent proteomics studies.

In one embodiment, a simple greedy algorithm may be used in which to solve the minimum missed peptide problem. The algorithm assigns identified peptides to proteins in the order of their detectabilities and does not change the peptide assignments once they are made. The algorithm assigns the peptide with lowest detectability first (denoted as Lowest-Detectability First Algorithm, LDFA). It should be noted that in LDFA, the assumption is made that the detectabilities of a single peptide in different parent proteins are close so that all identified peptides can be sorted based on their detectabilities. For comparison with LDFA, a greedy solution was implemented to the minimum protein set algorithm (GMPSA), which can be formulated as a set-covering problem with very little modification.

The performance of the LDFA and GMPSA was compared. First, identified peptides from a synthetic sample mixture B and Swiss-Prot as a reference database to conduct a controlled protein inference experiment was used. One advantage of this evaluation for quantifying the performance of the algorithm is that all proteins present in the sample are known. The sample mixture B contained 12 proteins corresponding to the 93 peptides identified in the experiment.

Out of 176,470 proteins from Swiss-Prot, 494 proteins (including the 12 proteins from the mixture) were identified as containing at least one identified peptide. The LDFA identified 12 proteins in the sample, 11 correctly. Of the 11 proteins that were correctly assigned, in only one instance could the algorithm not distinguish between the correct protein and one of its close homologs. This situation is defined herein as a “tie”. Each tie is resolved by random selection.

The same data was tested using the GMPSA, which tries to explain the identified peptides with the smallest possible number of proteins. GMPSA also identified 12 proteins as the total number of proteins in the sample, however, it suffered in accuracy. For 5 out of the 12 proteins, the GMPSA could not distinguish between the correct proteins and their homologs. Since in each step, the GMPSA considers only the number of the identified peptides per protein it is much more likely to encounter ties than the LDFA. As shown in FIG. 4, the GMPSA does not have the means of differentiating between proteins containing the same number of identified peptides. In practical situations this results in ties involving more homologs than the LDFA, thus reducing the chance of selecting the correct protein. An example of such a tie involves HBB_HUMAN. The LDFA found two possible solutions (HBB_HUMAN and HBB_GORGO), resulting in a 50% chance of a correct selection. On the other hand, the GMPSA selected between four different proteins (HBB_HUMAN, HBB_HAPGR, HBB_HYLLA and HBB_PANPO) resulting in 25% chance of a correct prediction. Furthermore, the smaller average number of proteins per tie encountered by LDFA is advantageous for reporting results of identification. To avoid information leak in calculating peptide detectabilities, the training set for the predictor was constructed from a different synthetic dataset.

The one protein that was not identified correctly by the LDFA, bovine RNase A, was assigned to a close homolog from one of 7 organisms (69.4% average sequence identity) chosen at random. This assignment was made with a single identified peptide. Furthermore, the sequence for bovine RNase A in the Swiss-Prot database includes the 26-amino acid signal peptide that is not actually present in the sample. Since LDFA takes into consideration the detectabilities of both identified and unidentified peptides, the presence of the signal peptide in the database hinders the assignment of bovine RNase A. After the signal peptide is removed, the sequence identity compared to all seven sequences that match the identified peptide is 84.0%. In comparison, the GMPSA randomly selects among 20 proteins from Swiss-Prot sharing the identified peptide.

Another illustrative experiment was performed on a biological sample from R. norvegicus, in which the correct proteins were not known. The identified peptides in the sample (693 in total) were searched against an IPI database located at http://ncbi.nlm.nih.gov and were found in 805 proteins. These are the proteins that may potentially be present in the sample. Table 5 shows the distribution of these peptides contained by different numbers of proteins. In this experiment, about 60% identified peptides (397 out of 693) are degenerate peptides, i.e. contained by two or more proteins. The two algorithms described above, LDFA and GMPSA, were run on this set.

TABLE 5

| | | | | | |
|---|---|---|---|---|---|
| # proteins | 1 | 2-5 | 6-10 | 11-20 | >20 |
| # peptides | 296 | 330 | 43 | 16 | 8 |

Mascot had originally assigned 301 proteins in this sample, LDFA assigned 275 proteins and GMPSA assigned 247 proteins. Taking into consideration all unique peptides from the rat sample only 149 proteins could be assigned by at least one unique peptide. Thus, any other protein to be assigned by any of the three methods would have to rely solely on degenerate peptides. Due to the prevalence of ties, GMPSA was run 30 times. Only 153 proteins were consistently assigned in all runs. Out of 430 proteins assigned over all GMPSA runs, 229 were assigned less than 50% of the time.

Since the correct proteins in this sample were not known, the accuracy of the LDFA and GMPSA could not be quantified as on the synthetic data. Instead, a different approach was taken where protein distinguishability was measured in this experiment. FIGS. 5(a)-(b) show all pairs of 805 identified proteins that shared at least one identified peptide. The y-axis corresponds to the percentage of sequence identity, while the x-axis represents the length of one of the proteins in the pair. FIG. 5(a) shows, through the solid triangles, all pairs of proteins that share at least one identified peptide and that the LDFA could not distinguish. This means that at one point during the execution, the LDFA had to randomly select between those two proteins and that at the completion of the algorithm one of the proteins is not present in the final solution. FIG. 5(b) shows the equivalent plot for the GMPSA. In a single run of each algorithm, there were 94 indistinguishable pairs for the LDFA and 2,346 indistinguishable pairs for the GMPSA. The total number of proteins that were excluded from the final solution at random was 69 and 188 for the LDFA and GMPSA, respectively.

In the previously described illustrative experiment, the Minimum acceptable Detectability of Identified Peptides (MDIP) was defined as the detectability of an identified peptide that maximizes the average of the true positive and true negative rates for an identified protein. Also shown was that MDIP of a protein is correlated with its abundance in the sample. The relationship between MDIP and MDAP is shown in FIG. 6 where the identified and non-identified peptides are shown for the same protein under two different experiments. While MDAP is the lowest detectability of an identified peptide in a protein, MDIP is influenced by non-identified peptides as well. Ideally, as in the left part of FIG. 6, peptides are consecutively identified according to their decreasing detectabilities (starting from the top one), thus giving MDIP=MDAP. Non-identified peptides in the right part of FIG. 4 allow discrepancy between these two quantities, which are believed to be useful for the advancement of label-free protein quantification.

When the same peptide can be assigned to multiple proteins, this task—referred to as the protein inference problem—is non-trivial. This problem was addressed by utilizing the concept of peptide detectability—the probability that a peptide will be identified in a shotgun proteomics experiment based on inherent properties of the peptide and its surroundings within a protein. As previously discussed, the rules governing peptide detectability can be assigned using a statistical inference model approach and that a peptide's detectability depends on its source protein concentration. In cases where a peptide sequence is found in multiple protein sequences, knowledge of the detectabilities of both the identified peptides (similar sequences in the multiple proteins) and the unidentified peptides (some of which will differ in the multiple proteins) can be used to discern between assignments that would not otherwise be distinguishable.

The exemplary results shown here for 766 peptides identified from a rat brain sample indicate that 247 proteins can be assigned using a greedy algorithm for the minimum protein coverage formulation, but 94 (38%) of these are selected randomly. When peptide detectability is incorporated into the assignment algorithm, 275 proteins are assigned and only 51 (19%) of these are ambiguous.

### Bayesian Modeling

In one illustrative method, the protein inference problem may be addressed by implementing two Bayesian models that take as input a set of identified peptides from any peptide search engine, and attempt to determine a most likely set of proteins from which those identified peptides originated. The first model assumes that all identified peptides are correct, whereas the second model also accepts the probability of each peptide to be present in the sample.

For purposes of illustrating the challenge of protein inference, a protein configuration graph is shown in FIG. 7(a), i.e. a bipartite graph in which two disjoint sets of vertices represent the proteins in the database and the peptides from these proteins, respectively, and where each edge indicates that the peptide belongs to the protein. The protein configuration graphs of FIGS. 7(a)-(c) is independent of the proteomics experiment, and thus can be built solely from a set, i.e., database, of protein sequences. Furthermore, the bipartite graph of FIGS. 7(a)-(c) considers the non-identified peptides. The protein configuration graph is partitioned into connected components, each representing a group of proteins (e.g., homologous protein families) sharing one or more (degenerate) peptides. If there are no degenerate peptides in the database, each connected component will contain exactly one protein and its peptides. In practice, however, the protein configuration graph may contain large connected components, especially for protein databases of higher animals or those containing closely related species.

Given that the protein-peptide bipartite graph may be interpreted as a Bayesian network with edges pointing from proteins into peptides, it is straightforward to show that protein inference can be addressed separately for each individual connected component. In this approach, the peptide identification results are first mapped to the protein configuration graph. A vector of indicator variables (y₁, . . . , yj, . . . , yₙ) is used, referred to herein as the peptide configuration to denote a set of identified peptides. Given the peptide configuration, a connected component of the protein configuration graph referred to as “trivial” if it contains no identified peptides. Clearly, in the case of trivial components protein inference is simple-none of the proteins should be present in the sample. Therefore, the protein inference problem may be reduced to finding the most likely protein configuration (x₁, . . . , xᵢ, . . . , xₘ) by analyzing non-trivial components only. In the first model, all identified peptides are assigned equal probabilities (i.e., =1) as shown in FIGS. 7(a)-(c), whereas in the advanced model different probabilities are considered for different identified peptides depending on the associated identification scores (s₁, . . . , sj, . . . , sₙ) (see FIG. 7(c)). Notation and definitions used herein are summarized in Table 6.

TABLE 6

| Notation | Definition |
|---|---|
| (1, . . . , i, . . . m) | m proteins within a non-trivial connected component of the protein configuration graph |
| (x₁, . . . xᵢ, . . . , xₘ) | protein configuration: a vector of indicator variables of proteins' presences |
| (1, . . . , j, . . . n) | all n peptides from m proteins being considered |
| (z₁₁, . . . zᵢⱼ, . . . , zₘₙ) | indicator variables of peptide j belonging to protein i |
| | if peptide j is a peptide from protein i, Zᵢⱼ = 1; |
| | otherwise Zᵢⱼ = 0 |
| (y₁, . . . yᵢ, . . . , yₙ) | peptide configuration: a vector of indicator variables of peptide being identified if peptide |
| | j is identified, yⱼ = 1; otherwise yⱼ = 0 |
| (s₁, . . . sᵢ, . . . , sₙ) | assigned scores of peptides |
| | if peptide j is not identified (i.e. yⱼ = 0), sⱼ = 0 |
| (r₁, . . . rᵢ, . . . , rₙ) | probabilities of peptide being correctly identified |
| | also the probabilities of peptides' presences |
| (LR₁, . . . LRᵢ, . . . , LRₙ) | likelihood ratio between peptides' presences and absences |
| (d₁₁, . . . dᵢⱼ, . . . , dₘₙ) | prior probabilities of peptides to be identified from proteins |
| | if Zᵢⱼ = 1, dᵢⱼ = the detectability of peptide j from protein i; |
| | otherwise, dᵢⱼ = 0 |

### First Bayesian Model

In this model, it is assumed that each identified peptide has an equally high prior probability to be present in the sample and low false discovery rate (FDR) in the results of peptide identification. In practice, even though this assumption does not completely hold, peptide FDRs are typically controlled at a low level (e.g., 0.01) by either a heuristic target-decoy search strategy or by probabilistic modeling of random peptide identification scores.

Consider m proteins and n peptides from these proteins within a non-trivial connected component of the protein configuration graph. Each protein i is either present in the sample or absent from it, which can be represented by an indicator variable xᵢ. Therefore, any solution of the protein inference problem corresponds to a vector of indicator variables, (x₁, . . . , xₘ), referred to as a protein configuration. Given the set of identified peptides from peptide search engines (peptide configuration (y₁, . . . , yₙ)), a goal is to find the maximum a posteriori (MAP) protein configuration, that is the configuration that maximizes the posterior probability P(x₁, . . . , xₘ|y₁, . . . , yₙ). Using the Bayes' rule, this posterior probability can be expressed as

$$P ( {x}_{1} , … , {x}_{m} \vert {y}_{1} , … , {y}_{n} ) = \frac{\begin{matrix}P ( {x}_{1} , … , {x}_{m} ) \\ P ( {y}_{1} , … , {y}_{n} \vert {x}_{1} , … , {x}_{m} )\end{matrix}}{{\sum }_{( {x}_{1} , … , {x}_{m} )} [ \begin{matrix}P ( {x}_{1} , … , {x}_{m} ) \\ P ( {y}_{1} , … , {y}_{n} \vert {x}_{1} , … , {x}_{m} )\end{matrix} ]} = \frac{P ( {x}_{1} , … , {x}_{m} ) {\prod }_{j} [ \begin{matrix}1 - {Pr ( {y}_{j} = 1 \vert {x}_{1} , … , {x}_{m} )}^{1 - {y}_{j}} \\ {Pr ( {y}_{j} = 1 \vert {x}_{1} , … , {x}_{m} )}^{{y}_{j}}\end{matrix} ]}{\begin{matrix}{\sum }_{( {x}_{1} , … , {x}_{m} )} P ( {x}_{1} , … , {x}_{m} ) \\ {\prod }_{j} [ \begin{matrix}1 - {Pr ( {y}_{j} = 1 \vert {x}_{1} , … , {x}_{m} )}^{1 - {y}_{j}} \\ {Pr ( {y}_{j} = 1 \vert {x}_{1} , … , {x}_{m} )}^{{y}_{j}}\end{matrix} ]\end{matrix}} \qquad ( 1 )$$

where P(x₁, . . . , xₘ) is the prior probability of the protein configuration. Assuming the presence of each protein i is independent of other proteins, this prior probability can be computed as

$$P ( {x}_{1} , … , {x}_{m} ) = {\prod }_{i} P ( {x}_{i} ) \qquad ( 2 )$$

Pr(yⱼ=1|x₁, . . . , xₘ) is the probability of peptide to be identified by shotgun proteomics given the protein configuration (x₁, . . . , xₘ). Assuming that different proteins are present in the sample independently of one another and ignoring the competition of peptides for ionization and MS/MS fragmentation, Pr(yⱼ=1|x₁, . . . , xₘ) may be computed as

$$Pr ( {y}_{j} = 1 \vert {x}_{1} , … , {x}_{m} ) = 1 - {\prod }_{i} [ 1 - {x}_{i} Pr ( {y}_{j} = 1 \vert {x}_{i} = 1 , {x}_{1} = … {x}_{i - 1} = {x}_{i + 1} = … {x}_{m} = 0 ) ] \qquad ( 3 )$$

where Pr(yⱼ=1|x₁=1,x₁= . . . xᵢ₋₁=xᵢ₊₁=xₘ=0) is the probability of peptide j to be identified if only protein i is present in the sample. This probability is further expressed as

$$Pr ( {y}_{j} = 1 \vert {x}_{i} = 1 , {x}_{1} = … {x}_{i - 1} = {x}_{i + 1} = … {x}_{m} = 0 ) = \{ \begin{matrix}0 & if {Z}_{ij} = 0 \\ {d}_{ij} & otherwise\end{matrix} \qquad ( 4 )$$

As previously shown, for a particular proteomics platform (e.g., LC-MS/MS), this probability, referred to as the standard peptide detectability, is an intrinsic property of the peptide (within its parent protein), and may be predicted from the peptide and protein sequence prior to a proteomics experiment. For simplicity, the definitions dᵢⱼ=0 if Zᵢⱼ=0 (see Table 8) are provided. Combining equations above, the posterior probabilities for protein configurations may be determined as

$$P ( {x}_{1} , … , {x}_{m} \vert {y}_{1} , … , {y}_{n} ) = \frac{{\prod }_{i} P ( {x}_{i} ) {\prod }_{j} \{ \begin{matrix}{[ {\prod }_{i} ( 1 - {x}_{i} {d}_{ij} ) ]}^{1 - {y}_{j}} \\ {[ 1 - {\prod }_{i} ( 1 - {x}_{i} {d}_{ij} ) ]}^{{y}_{j}}\end{matrix} \}}{{\sum }_{( {x}_{1} , … , {x}_{m} )} {\prod }_{i} P ( {x}_{i} ) {\prod }_{j} \{ \begin{matrix}{[ {\prod }_{i} ( 1 - {x}_{i} {d}_{ij} ) ]}^{1 - {y}_{j}} \\ {[ 1 - {\prod }_{i} ( 1 - {x}_{i} {d}_{ij} ) ]}^{{y}_{j}}\end{matrix} \}} \qquad ( 5 )$$

Hence, protein inference is equivalent to finding the MAP protein configuration maximizing the above function

(x₁^{max}, . . . , xₘ^{max})=aug max₍ₓ_{₁}_{, . . . , x}_{ₘ}₎P(x₁, . . . , xₘ|y₁, . . . , yₙ) (6)

The posterior probability of a specific protein i to be present in the sample may be derived from eqn. (6) as

$$\begin{matrix}{P}^{o} ( {x}_{i} ) = P ( {x}_{i} \vert {y}_{1} , … , {y}_{n} ) \\ = {\sum }_{{x}_{1} , … , {x}_{i - 1} , {x}_{i + 1} , … , {x}_{m}} P ( {x}_{1} , … , {x}_{m} \vert {y}_{1} , … , {y}_{n} )\end{matrix} \qquad ( 7 )$$

Similarly, the posterior probability of a peptide j can be computed as

$$\begin{matrix}P ( {y}_{j} ) = {\sum }_{( {x}_{1} , … , {x}_{m} )} [ P ( {y}_{j} \vert {x}_{1} , … , {x}_{m} ) P ( {x}_{1} , … , {x}_{m} ) ] \\ = {[ 1 - {\prod }_{i} ( 1 - Pr ( {x}_{i} = 1 ) {d}_{ij} ) ]}^{{y}_{j}} \\ {[ {\prod }_{i} ( 1 - Pr ( {x}_{i} = 1 ) {d}_{ij} ) ]}^{1 - {y}_{j}}\end{matrix} \qquad ( 8 )$$

### Second Bayesian Model

The first model described above assumes all identified peptides have equal probability (=1) of being correctly identified. In the second model, this assumption may be relaxed by introducing a peptide identification score sⱼ for each peptide j, which is outputted by peptide search engines. It is assumed that the peptide identification score is highly correlated with the probability of a peptide being correctly identified and their relationship (denoted by rⱼ=Pr(yⱼ=1|sⱼ)) may be approximately modeled using probabilistic methods adopted by some search engines such as Mascot or post-processing programs such as PeptideProphet. This allows the determination P(x₁, . . . , xₘ|s₁, . . . , sₙ) by enumerating all potential peptide configurations

$$\begin{matrix}P ( {x}_{1} , … , {x}_{m} \vert {s}_{1} , … , {s}_{n} ) = {\sum }_{( {y}_{1} , … , {y}_{n} )} [ \begin{matrix}P ( {x}_{1} , … , {x}_{m} ; {y}_{1} , … , {y}_{n} ) \\ P ( {s}_{1} , … , {s}_{n} )\end{matrix} ] \\ = {\sum }_{( {y}_{1} , … , {y}_{n} )} [ \begin{matrix}P ( {x}_{1} , … , {x}_{m} \vert {y}_{1} , … , {y}_{n} ) \\ P ( {y}_{1} , … , {y}_{n} \vert {s}_{1} , {…s}_{n} )\end{matrix} ] \\ = {\sum }_{( {y}_{1} , … , {y}_{n} )} [ \begin{matrix}\frac{P ( {x}_{1} , … , {x}_{m} )}{P ( {s}_{1} , … , {s}_{n} )} \\ P ( {y}_{1} , … , {y}_{n} \vert {x}_{1} , … {x}_{m} ) \\ P ( {s}_{1} , … , {s}_{n} \vert {y}_{1} , … , {y}_{n} )\end{matrix} ]\end{matrix} \qquad ( 9 )$$

Assuming that sⱼ is independent of the presence of the other peptides (i.e. (y₁, . . . , yⱼ₋₁, yⱼ₊₁, . . . , yₙ)) for each peptide j, it is provided that

$$P ( {s}_{1} , … , {s}_{n} \vert {y}_{1} , … , {y}_{n} ) = {\prod }_{j} P ( {s}_{j} \vert {y}_{j} ) \qquad ( 10 )$$

Applying Bayes' rule provides

$$\begin{matrix}P ( {s}_{1} , … , {s}_{n} \vert {y}_{1} , … , {y}_{n} ) = {\prod }_{j} \frac{P ( {y}_{j} \vert {s}_{j} ) P ( {s}_{j} )}{P ( {y}_{j} )} \\ = {\prod }_{j} \frac{{( 1 - {r}_{j} )}^{1 - {y}_{j}} {r}_{j}^{{y}_{j}} P ( {s}_{j} )}{P ( {y}_{j} )}\end{matrix} \qquad ( 11 )$$

Combining eqns. 5 to 11, the posterior probability of protein configurations may be found as

$$P ( {x}_{1} , … , {x}_{m} \vert {s}_{1} , … , {s}_{n} ) = \frac{{\sum }_{( {y}_{1} , … , {y}_{n} )} \{ {\prod }_{i} P ( {x}_{i} ) {\prod }_{j} \{ \begin{matrix}{[ {\prod }_{i} ( 1 - {x}_{i} {d}_{ij} ) ]}^{1 - {y}_{j}} \\ {[ 1 - {\prod }_{i} ( 1 - {x}_{i} {d}_{ij} ) ]}^{{y}_{j}} \\ \frac{{( 1 - {r}_{j} )}^{1 - {y}_{j}} {r}_{j}^{{y}_{j}}}{P ( {y}_{j} )}\end{matrix} \} \}}{{\sum }_{( {x}_{1} , … , {x}_{m} ; {y}_{1} , … , {y}_{n} )} \{ {\prod }_{i} P ( {x}_{i} ) {\prod }_{j} \{ \begin{matrix}{[ {\prod }_{i} ( 1 - {x}_{i} {d}_{ij} ) ]}^{1 - {y}_{j}} \\ {[ 1 - {\prod }_{i} ( 1 - {x}_{i} {d}_{ij} ) ]}^{{y}_{j}} \\ \frac{{( 1 - {r}_{j} )}^{1 - {y}_{j}} {r}_{j}^{{y}_{j}}}{P ( {y}_{j} )}\end{matrix} \} \}} \qquad ( 12 )$$

In the Bayesian models, no prior knowledge about the protein presence in the sample is assumed. Therefore, in eqns. 5 and 12, P(xᵢ) is regarded as constant (i.e., Pr(xᵢ=1)=0.5) for all proteins. In practice, prior knowledge, such as the species which the sample is from, the size of entire protein database, known protein relative quantities or protein families that are likely present in the sample, may be directly integrated into the Bayesian models.

Similarly to the basic model, the posterior probability of a specific protein i present in the sample may be computed as

$$P ( {x}_{i} \vert {s}_{1} , … , {s}_{n} ) = {\sum }_{( \begin{matrix}{x}_{1} , … , {x}_{i - 1} , \\ {x}_{i + 1} , {… }_{n} {x}_{m}\end{matrix} )} P ( {x}_{1} , … , {x}_{m} \vert {s}_{1} , … , {s}_{n} ) \qquad ( 13 )$$

and the posterior probability of a peptide j in the sample.

An adjustment of the predicted peptide detectabilities is typically necessary when applying them in eqn. 4, since the predicted peptide detectabilities (denoted as d⁰ᵢⱼ) reflect the detectability of a peptide under a standard proteomics experimental setting, in particular, under fixed and equal abundances (i.e., q⁰) for all proteins. Therefore, the following illustrative method is utilized to adjust the standard peptide detectabilities in a given experiment.

Assuming that the abundance of protein i in the sample mixture is qᵢ instead of q⁰, the effective detectability of peptide j from this protein should be adjusted to

dᵢⱼ=1−(1−dᵢⱼ⁰)^{q}^{ᵢ}^{/q}^{⁰} (14)

Although qᵢ is not explicitly known, since the total probability of observing a peptide j is given by rⱼ (or yⱼ for basic model), qᵢ may be estimated by solving the equation Σⱼdij=ΣⱼZᵢⱼrⱼ for a specific protein i. This adjustment method may be utilized to adjust the predicted peptide detectabilities based on the estimated protein abundances.

Given a protein configuration graph, such as that shown in FIGS. 7(a)-(c), the peptide detectabilities (dᵢⱼ) and the probabilities of peptide presence in the sample (rⱼ), the posterior distribution of protein configurations may be computed directly from eqns. 5 or 12, depending on which Bayesian model (first or second) is used. This brute force method is, however, typically expensive and may only works for small connected components in the protein configuration graph, since it requires computing the summation over all potential protein configurations, which has computational complexity of O(2^{m}).

Gibbs sampling is a strategy that may be used to rapidly approximate a high dimensional joint distribution that is not explicitly known. The Gibbs sampling algorithm was implemented in the illustrative methods implementing the first and second Bayesian models to achieve the optimal protein configuration with the MAP probability. The original Gibbs sampling algorithm considers one individual variable at a time in the multi-dimensional distribution. It, however, often converges slowly and is easily trapped by local maxima. Several techniques have been proposed to optimize the search efficiency of a Gibbs sampling algorithm, such as random sweeping, blocking and collapsing. Because in this case each variable xᵢ to be sampled has small search space (i.e. {0,1}), the blocking sampling technique was implemented in a Gibbs sampler algorithm described herein. It should be appreciated, however, that the other techniques discussed herein may be implemented for optimization purposes. The first Bayesian model may serve as an example to illustrate the memorizing technique. For a selected t-block in the protein configuration, Bₓ=(xᵥ_{₁}, . . . , xᵥ_{ₜ}), where 1≦vₖ≦m for k=1, . . . , t, a t-block function is defined by

$$F ( {x}_{{v}_{1}}^{′} , … , {x}_{{v}_{t}}^{′} ) = \frac{{\prod }_{k} P ( {x}_{{v}_{k}} ) {\prod }_{j} { }_{j , i ∈ \{ {v}_{1} , … , {v}_{t} \}} {\{ \begin{matrix}{[ {\prod }_{i} ( 1 - {x}_{i} {d}_{ij} ) ]}^{1 - {y}_{j}} \\ {[ 1 - {\prod }_{i} ( 1 - {x}_{i} {d}_{ij} ) ]}^{{y}_{j}}\end{matrix} \}}^{{Z}_{ij}}}{{\sum }_{( {x}_{{v}_{1}}^{′} , … , {x}_{{v}_{t}}^{′} )} \{ \begin{matrix}{\prod }_{k} P ( {x}_{{v}_{k}} ) {\prod }_{j} { }_{j , i ∈ \{ {v}_{1} , … , {v}_{t} \}} \\ {\{ \begin{matrix}{[ {\prod }_{i} ( 1 - {x}_{i} {d}_{ij} ) ]}^{1 - {y}_{j}} \\ {[ 1 - {\prod }_{i} ( 1 - {x}_{i} {d}_{ij} ) ]}^{{y}_{j}}\end{matrix} \}}^{{Z}_{ij}}\end{matrix} \}} \qquad ( 15 )$$

where the indicator variables xₖ are inherited from previous sampling procedure. The posterior probability of the updated configuration is

P(x₁, . . . , xᵥ_{₁}⁻¹,x′ᵥ_{₁},xᵥ_{₁}₊₁, . . . xᵥ_{ₖ}₋₁,x′ᵥ_{ₖ},sᵥ_{ₖ}₊₁, . . . , xₘ|y₁, . . . , yₙ)=T×F(x′ᵥ_{₁}, . . . , x′ᵥ_{ₜ}) (16)

where the normalizing factor T may be computed from inherited variables xᵥ_{ₖ}, and thus need to be computed only once for all (xᵥ_{₁}, . . . xᵥ_{ₜ}) to be evaluated in each sampling step. Therefore, without increasing the computational complexity, a memorizing strategy is adopted that keeps a record of all (as well as the maximum) posterior probabilities (and the corresponding protein configurations) among all configurations evaluated during the sampling procedure, and reports the maximum solution in the end. The memorized posterior probabilities may also be used to calculate the marginal protein posterior probabilities in eqns. 7 and 13.

In one illustrative procedure, two datasets from different sources that are both generated using mixtures of model proteins were used. Therefore, the proteins in these samples are known. A first dataset is used only for the training of the detectability predictor, while the second dataset was used for testing the protein inference methods. The first data set from a mixture (Sample A) of 13 standard proteins was prepared at 1 μM final digestion concentration for each protein except human hemoglobin which is at 2 μM, combined with buffer, reduced, alkylated, and digested overnight with trypsin. Peptides were separated by nano-flow reversed-phase liquid chromatography gradient and analyzed by mass spectrometry and tandem mass spectrometry. In one embodiment these spectrometric procedures may be performed using a ThermoElectron (SanJose, Calif.) LTQ linearion trap mass spectrometer. The second mixture (SampleSigma49) was cleaned up by gel electrophoresis, reduced, alkylated, and digested in-gel with trypsin. Tandem mass spectra for doubly-charged precursor ions were obtained from a website at Vanderbilt University and searched against human sequences in Swiss-Prot using Sequest.

In the illustrative methods described herein, the first and second Bayesian models described in were tested on the Sigma49 sample. The peptide detectability predictors were trained using Sample A following the method described in Experiment #1 herein. Similarly to that previously described as in, prior to the protein inference, 13388 MS/MS spectra acquired from Sigma49 sample in one LC/MS experiment were searched against the human proteome in Swiss-Protdatabase (version 54.2 in this illustrative experiment). PeptideProphet was then used to assign a probability score for each identified peptide. For the first Bayesian model, 152 unique peptides with minimum PeptideProphet probability score 0.95 were retained as identified peptides, while for the advanced model, 443 peptides were retained with minimum probability score 0.05. Two methods were preformed to set the prior probability rⱼ for each identified peptide. In the first method, the probability for each identified peptide reported by PeptideProphet was used. Since PeptideProphet does not consider peptide detectability, the second method was implemented, which converts the PeptideProphet probability into a likelihood ratio LRⱼ and then apply eqn. 10. The conversion may be done by LRⱼ=P_{rPP} (yⱼ=1)/[c×(1−P_{rPP} (yⱼ=1))], where P_{rPP} (yⱼ=1) is the PeptideProphet probability, and c is the ratio between the prior probabilities of the peptide's presence and absence. For both models, block size 3 in Gibbs sampler was used.

Table 7 compares the results from the first and second Bayesian models with that from ProteinProphet and the minimum missed peptide (MMP) approach previously mentioned on the Sigma49 sample. Sigma49 sample was prepared by mixing 49 human proteins, among which 44 proteins contain at least one peptide that can be identified by shotgun proteomics. In addition, 9 keratin proteins and 4 other proteins are categorized as the “keratin contamination” and “bonus” proteins, respectively, and were believed to be present in the sample due to contamination.

TABLE 7

| | MMP | PP | BB | BBA | ABP | ABPA | ABL | ABLA |
|---|---|---|---|---|---|---|---|---|
| TP | 39/41/45 | 41.5/43.5/47.5 | 39/41/47 | 37/39/43 | 35/35/39 | 43/45/49 | 37/38/41 | 44/46/50 |
| FP | 6/4/0 | 7.5/1.5/5.5 | 16/14/8 | 6/4/0 | 4/4/0 | 22/20/16 | 4/3/0 | 9/7/3 |
| FN | 5/7/12 | 2.5/4.5/9.5 | 5/7/10 | 7/9/14 | 9/13/18 | 1/3/8 | 7/10/16 | 0/3/7 |
| PR | 0.87/0.91/1 | 0.85/0.89/0.07 | 0.71/0.75/0.35 | 0.86/0.01/1 | 0.9/0.0/1 | 0.66/0.69/0.75 | 0.0/0.93/1 | 0.83/0.87/0.94 |
| RC | 0.89/0.85/0.79 | 0.94/0.91/0.89 | 0.89/0.85/0.82 | 0.84/0.81/0.75 | 0.8/0.73/0.68 | 0.98/0.94/0.86 | 0.84/0.79/0.72 | 1/0.96/0.88 |
| F | 0.88/0.88/0.88 | 0.89/0.90/0.90 | 0.79/0.8/0.84 | 0.85/0.86/0.86 | 0.84/0.8/0.81 | 0.79/0.80/0.81 | 0.87/0.85/0.84 | 0.91/0.91/0.91 |

TP = True Positive
FP = False Positive
FN = False negative number of proteins
PR = Precision
RC = Recall
F = F-measure in 3 categories of proteins in the sample
MMP = Minimum missed peptide
PP = ProteinProphet
BB = First Bayesian model
BBA = First Bayesian model with detectability adjustment
ABP = Second Bayesian model w/ raw PeptideProphet probability
ABPA = ABP after detectability adjustment
ABL = Second Bayesian model using converted probabilty score
ABLA = ABL after detectability adjustment

From the results, it is observed that the incorporation of detectabilities into the PeptideProphet probability improves the performance of the probabilistic models (e.g., ABLA vs. ABPA), which indicates the peptide detectability is a useful concept in estimating peptide probability and protein inference. The detectability adjustment also improves accuracy (expressed by the F measure) of the protein inference (e.g., BBA vs. BB or ABLA vs. ABL), implying that the predicted peptide detectabilities need to be corrected by peptide quantities in real proteomics experiments. Overall, ABLA model performs better than the other methods. Surprisingly, simple approaches like MMP or ProteinProphet can achieve comparable accuracy as ABLA. However, it is hard to draw a firm conclusion from the comparison on a relatively simple protein mixture. Further comparative analysis of these models is needed to use more complex (e.g. with hundreds of proteins), but well elucidated samples like Sigma49.

FIG. 8 illustrates the results of ABLA on 5 connected components in the protein configuration graph built from Sigma49 dataset. The model proteins and likely contaminated proteins in the sample received higher marginal posterior probabilities than the other proteins, and the MAP configuration contains mostly true proteins. PeptideProphet cannot resolve the correct protein assignment in component A and C. It is noted that component A consists of three proteins (P51965, Q96LR5 and Q969T4) which share only one identified peptide. ABLA algorithm correctly assigns the true model protein (P51965) as the MAP configuration over the other two proteins.

In this study a new methodology is proposed and evaluated for protein inference in shotgun proteomics. The two models proposed herein are based on Bayesian inference in which the solution is the set of proteins that is most likely to be present in the sample. The new approach has three advantages over the existing methods: (1) It calculates or, if global optimum is not reached, approximates a MAP solution for the set of proteins present in the sample and can also output the probability of each protein to be present in the sample; (2) It can output the posterior probabilities of the identified peptides to be present in the sample, given the entire experiment. Since these modified probabilities should better reflect the overall experimental results, the overall peptide identification may be improved; (3) The Gibbs sampling approach used to approximate the posterior probabilities of protein configuration is a proven methodology, and its performance and convergence has been well-studied.

It is common in proteomics for a sample to be analyzed multiple times in order to increase coverage of the proteome and/or to increase confidence in low sequence coverage proteins. While not specifically addressed, the application of the Bayesian models described herein adequately accommodates such data since peptide detectability, used to calculate prior probabilities, should assign lower values to those peptides not identified in all the replicate analyses. In addition, higher mammals often contain multiple very similar homologous proteins due to recent gene duplications. These proteins are almost impossible to differentiate using shotgun proteomics, if some but not all of these proteins are present in the sample. As a result, although the MAP protein configuration will contain at least one of these proteins, they each can receive a low marginal probability (e.g. <0.5). While this situation is not explicitly addressed here, it is noted that the models proposed above can be easily modified to consider a given set of proteins as a group and then compute the probability of their presence as a whole. We will test this functionality in future implementation of the models.

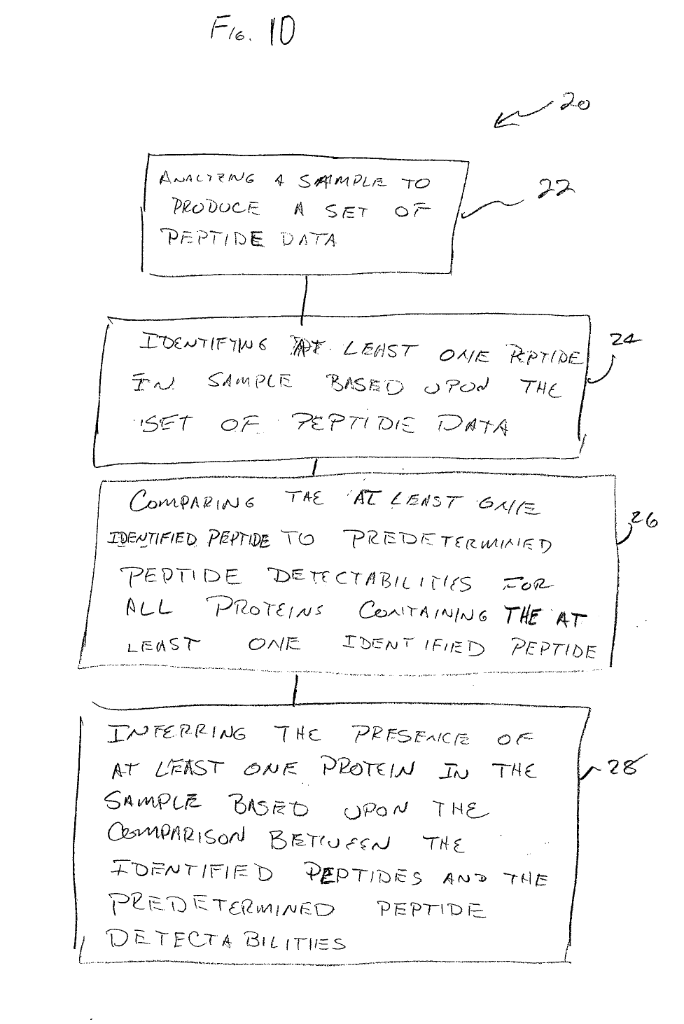

FIG. 9 shows a flowchart 10 illustrating a method for inferring the presence of a protein in a sample being analyzed, as has been described herein. The flowchart 10 includes operation 12, which may include entering a peptide training data set into a statistical inference model, which may be performed as described herein. Operation 14 may include training the statistical inference model with the peptide training data set, which may be performed as described herein. Operation 16 may include determining predicted detectability of at least one peptide present in a sample with the trained statistical inference model, which may be performed as described herein. Operation 18 may include inferring the presence of a protein in the sample based upon the determined detectability of the at least one peptide, which may be performed as described herein. It should be appreciated that operation 18 may include quantification of proteins present in the sample.

FIG. 10 shows another flowchart 20 illustrating another method for inferrring the presence of a protein in a sample being. Operation 22 may include analyzing a sample to produce a set of peptide data, which may be performed as described herein. Operation 24 may include identifying at least one peptide in the sample based upon the set of peptide data, which may be performed as described herein. Operation 26 may include comparing the at least one identified peptide to predetermined peptide detectabilities for all proteins containing the at least one identified peptide, which may be performed as described herein. Operation 28 may include inferring the presence of at least one protein in the sample based upon the comparison between the identified peptides and the predetermined peptide detectabilities. It should be appreciated that operation 28 may include quantification of proteins present in the sample.

It should be appreciated that the order of many of the steps of the methods described herein may be altered in both order and substance. Furthermore, one or more operations described herein may be removed in various methods or others included.

There are a plurality of advantages of the present disclosure arising from the various features of the methods described herein. It will be noted that alternative embodiments of the methods of the present disclosure may not include all of the features described yet still benefit from at least some of the advantages of such features. Those of ordinary skill in the art may readily devise their own implementations of methods that incorporate one or more of the features of the present disclosure and fall within the spirit and scope of the present disclosure.

1. A method of inferring presence of at least one protein in a sample, the method comprising:
entering a peptide training data set into a statistical inference model,
training the statistical inference model with the peptide training data set,
determining predicted detectability of at least one peptide present in the sample with the trained statistical inference model, and
inferring the presence of the at least one protein in the sample based upon the determined predicted detectability.

2. The method of claim 1, wherein the statistical inference model is a machine learning model.

3. The method of claim 2, wherein the entering the machine learning model is a support vector model system.

4. The method of claim 2, wherein the machine learning model is a supervised machine learning model.

5. The method of claim 2, wherein the machine learning model is a semi-supervised machine learning model.

6. The method of claim 1, wherein the statistical inference model is a neural network.

7. The method of claim 1, wherein the peptide training set includes a number of peptide features.

8. A method of quantifying proteins present in a sample, the method comprising:
entering a peptide training data set into a statistical inference model,
training the statistical inference model with the peptide training data set,
determining predicted detectability of at least one peptide present in the sample with the trained statistical inference model, and
quantifying proteins present in the sample with an analyzer based upon the determined predicted detectabilities.

9. The method of claim 8, wherein the statistical inference model is a machine learning model.

10. The method of claim 9, wherein the machine learning model is a supervised machine learning model.

11. The method of claim 9, wherein the machine learning model is a semi-supervised machine learning model.

12. The method of claim 8, wherein the statistical inference model is a neural network.

13. A method of inferring presence of at least one protein in a sample, the method comprising:
analyzing the sample to produce a set of peptide data,
identifying peptides in the sample based upon the peptide data,
comparing the identified peptides to predetermined peptide detectabilities for all proteins containing at least one identified peptides, and
inferring the presence of at least one protein in the sample based upon the comparison between the identified peptides and the predetermined peptide detectabilities.

14. The method of claim 13, wherein the inferring the presence of at least one protein based upon the comparison between the identified peptides and the predetermined peptide detectabilities comprises determining the probability of the presence of the at least one protein in the sample based upon the predetermined peptide detectabilities.

15. A method of quantifying at least one protein in a sample, the method comprising:
analyzing the sample to produce a set of peptide data,
identifying peptides in the sample based upon the peptide data,
comparing the identified peptides to predetermined peptide detectabilities for all proteins containing at least one identified peptides, and
quantifying at least one protein in the sample based upon the comparison between the identified peptides and the predetermined peptide detectabilities.
